(19) United States
(12) Patent Application Publication
(10) Pub. No.: US 2024/0120035 A1
(43) Pub. Date: Apr. 11, 2024

(54) CUTOFF ENERGY DETERMINATION METHOD AND INFORMATION PROCESSING DEVICE

(71) Applicant: Fujitsu Limited, Kawasaki-shi (JP)

(72) Inventor: Eiji OHTA, Yokohama (JP)

(73) Assignee: Fujitsu Limited, Kawasaki-shi (JP)

(21) Appl. No.: 18/221,394

(22) Filed: Jul. 13, 2023

(30) Foreign Application Priority Data
Oct. 5, 2022 (JP) 2022-160925

(51) Int. Cl. G16C 60/00; G06F 17/11; G16C 10/00

(52) U.S. Cl. CPC G16C 60/00; G06F 17/11; G16C 10/00

(57) ABSTRACT

A non-transitory computer-readable recording medium stores a program for causing a computer to execute a process, the process includes in repeat calculation of electron density of a substance by a self-consistent field method that uses a specific number of wave functions according to cutoff energy, executing a second electron density calculation at an (N+1)-th time (N is an integer greater than or equal to 1) by applying second cutoff energy of a value smaller than first cutoff energy applied to an first electron density calculation at an N-th time, determining whether the electron density obtained by the second electron density calculation at the (N+1)-th time satisfies a predetermined condition, and outputting the value of the second cutoff energy in a case where the condition is not satisfied.

### CROSS-REFERENCE TO RELATED APPLICATION

This application is based upon and claims the benefit of priority of the prior Japanese Patent Application No. 2022-160925, filed on Oct. 5, 2022, the entire contents of which are incorporated herein by reference.

### FIELD

The embodiments discussed herein are related to a cutoff energy determination method and an information processing device.

### BACKGROUND

In recent years, in the field of material development, materials informatics (hereinafter referred to as MI) has progressed worldwide as an approach of data-driven research and development aiming at shortening of a research and development period and cost reduction. Accumulation of high-quality material data is important for MI. Therefore, efficient data accumulation by simulation such as quantum chemistry is expected in addition to experiments.

Density functional theory (DFT) is one of main calculation methods of quantum chemistry. In the DFT, first, an electron density of a substance to be calculated is calculated by a self-consistent field (SCF) method. Then, a physical quantity (total energy or the like) indicating a state of the substance is calculated based on the obtained electron density.

As a technique using the DFT, for example, a method of designing a phase change-type optical recording medium has been proposed in which a material having desired optical characteristics is efficiently selected as a material of a recording layer when the phase change-type optical recording medium is designed.

Japanese Laid-open Patent Publication No. 10-334515 is disclosed as related art.

### SUMMARY

According to an aspect of the embodiments, a non-transitory computer-readable recording medium stores a program for causing a computer to execute a process, the process includes in repeat calculation of electron density of a substance by a self-consistent field method that uses a specific number of wave functions according to cutoff energy, executing a second electron density calculation at an (N+1)-th time (N is an integer greater than or equal to 1) by applying second cutoff energy of a value smaller than first cutoff energy applied to a first electron density calculation at an N-th time, determining whether the electron density obtained by the second electron density calculation at the (N+1)-th time satisfies a predetermined condition, and outputting the value of the second cutoff energy in a case where the condition is not satisfied.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a diagram illustrating an example of a cutoff energy determination method according to a first embodiment;

FIG. 2 is a diagram illustrating an example of a system configuration;

FIG. 3 is a diagram illustrating an example of a hardware configuration of a control node;

FIG. 4 is a diagram illustrating an example of a hardware configuration of a calculation node;

FIG. 5 is a diagram illustrating a relationship between Ecut and total energy;

FIG. 6 is a block diagram illustrating an example of functions implemented by a calculation system;

FIG. 7 is a flowchart illustrating an example of a substance state calculation procedure;

FIG. 8 is a diagram illustrating an example of differences and a representative value of differences to differenceₙ₋₁;

FIG. 9 is a flowchart illustrating an example of a procedure of Ecut determination processing;

FIG. 10 is a diagram illustrating a comparative example of a case where Ecut is fixed and a case where Ecut is varied;

FIG. 11 is a flowchart illustrating an example of an SCF calculation procedure; and

FIG. 12 is a diagram illustrating a relationship between Ecut and a calculation time.

### DESCRIPTION OF EMBODIMENTS

When the number of atoms is assumed to be N (N is a natural number), a calculation amount of the DFT is O(N³) in order notation, and the calculation amount becomes enormous when a system to be calculated becomes large. Therefore, in the DFT, cutoff energy is set as an important calculation condition for determining a resolution of calculation. The cutoff energy is a parameter related to the shortest wavelength of a wave of which a wave function is included in the calculation. As the cutoff energy is higher, a wave function of a wave having a shorter wavelength is included in the calculation. In a case where a setting value of the cutoff energy is inappropriate, the following problems occur.

In a case where the cutoff energy is too low, the total energy value is overestimated over a real value. In a case where the cutoff energy is excessive, the calculation resolution becomes more than needed, and an extra calculation time is required. Therefore, it is important to obtain optimal cutoff energy before performing detailed calculation of the physical quantity using the DFT.

In the past, for example, DFT calculation has been executed a plurality of times by changing (increasing or decreasing) the cutoff energy, and the minimum cutoff energy Ecut at which the total energy value converges has been determined as the optimal cutoff energy. The calculation time of such a plurality of times of DFT calculation is enormous, and it is required to shorten the calculation time.

Hereinafter, embodiments will be described with reference to the drawings. Note that the embodiments may be implemented in combination as long as no contradiction arises.

### First Embodiment

A first embodiment is a cutoff energy determination method capable of determining cutoff energy optimal for being applied to quantum chemistry calculation based on DFT, in a short time.

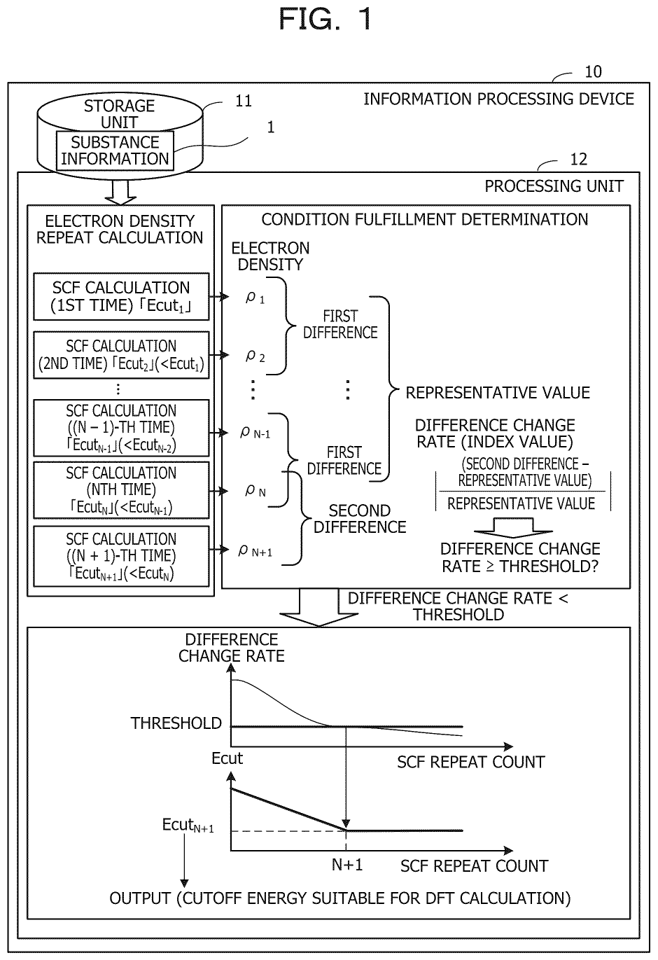

FIG. 1 is a diagram illustrating an example of a cutoff energy determination method according to the first embodiment. FIG. 1 illustrates an information processing device 10 that implements the cutoff energy determination method. The information processing device 10 may implement the cutoff energy determination method by, for example, executing a cutoff energy determination program.

The information processing device 10 includes a storage unit 11 and a processing unit 12. The storage unit 11 is, for example, a memory or a storage device included in the information processing device 10. The processing unit 12 is, for example, a processor or an arithmetic circuit included in the information processing device 10.

The storage unit 11 stores substance information 1 regarding a substance to be analyzed by the DFT. The substance information includes, for example, information of atoms constituting the substance, the number of electrons, and the like.

The processing unit 12 determines cutoff energy to be used for quantum chemistry calculation by the DFT for the substance indicated in the substance information 1. For example, the processing unit 12 performs repeat calculation (SCF calculation) of electron density of the substance by a self-consistent field (SCF) method using a specific number of wave functions according to the cutoff energy.

In the repeat calculation of the electron density, the processing unit 12 executes a second electron density calculation at the (N+1)-th time (N is an integer of 1 or more) by applying second cutoff energy having a value smaller than first cutoff energy applied to a first electron density calculation at the N-th time. Note that the cutoff energy applied to the SCF calculation at the first time is a preset value. A sufficiently high value is set as the cutoff energy to be applied to the SCF calculation at the first time. The sufficiently high value is cutoff energy capable of causing total energy of the substance to converge to the minimum value.

For example, in a case where the cutoff energy used in the SCF calculation at the first time is Ecut₁ and the cutoff energy used in the SCF calculation at the second time is Ecut₂, Ecut₂ is a value smaller than Ecut₁ (Ecut₂<Ecut₁). For example, in a case where the cutoff energy used in the SCF calculation at the (N−1)-th time is Ecut_{N-1} and the cutoff energy used in the SCF calculation at the N-th time is Ecut_{N}, Ecut_{N} is a value smaller than Ecut_{N-1} (Ecut_{N}<Ecut_{N-1}). Similarly, in a case where the cutoff energy used in the SCF calculation at the (N+1)-th time is Ecut_{N+1}, Ecut_{N+1} is a value smaller than Ecut_{N} (Ecut_{N+1}<Ecut_{N}).

The processing unit 12 determines whether or not the electron density obtained by the second electron density calculation at the (N+1)-th time satisfies a predetermined condition. The predetermined condition is, for example, a condition that a difference change rate is equal to or greater than a threshold.

The difference change rate is calculated based on a first difference and a second difference. The first difference is a value indicating a difference between the electron density set at the start of calculation and the electron density obtained as a calculation result. The second difference is a value indicating a difference between the electron density set at the start of calculation in the second electron density calculation at the (N+1)-th time and the electron density obtained as a calculation result. The difference change rate is, for example, an absolute value of a quotient obtained by dividing a difference between a representative value (mean, median, or the like) of the first difference for each electron density calculation up to the electron density calculation at the N-th time and the second difference by the representative value.

In a case where the condition is not satisfied, the processing unit 12 outputs the value of the second cutoff energy. In a case of the condition that the difference change rate is equal to or greater than the threshold, the processing unit 12 determines that the condition is not satisfied and outputs the value of the second cutoff energy when the difference change rate is less than the threshold.

The output second cutoff energy is the optimal cutoff energy in the density functional theory (DFT). Therefore, the processing unit 12 applies, for example, the second cutoff energy, and calculates a predetermined physical quantity of the substance based on the DFT.

Note that, in a case where the condition is satisfied, the processing unit 12 executes the electron density calculation at the (N+2)-th time, applying third cutoff energy obtained by reducing a predetermined value from the second cutoff energy. By reducing the cutoff energy, the electron density obtained by electron density calculation eventually satisfies a predetermined condition.

As described above, by reducing the cutoff energy to be applied each time the SCF calculation is executed, the optimal cutoff energy may be obtained before the electron density by the SCF calculation converges. For example, when the cutoff energy to be applied to the SCF calculation is sufficiently high, the SCF calculation considering more waves is performed, and thus, the amount of difference change in the electron density by the calculation becomes a large value. By the time the cutoff energy is reduced to reach an optimal value (smallest value at which the minimum value of the total energy may be calculated), the amount of difference change in the electron density by the SCF calculation becomes a small value. Then, when the SCF cutoff energy becomes smaller than the optimal value, the cutoff energy is not able to reach the minimum value of the total energy, and thus the amount of difference change in the electron density due to repetition of the SCF calculation becomes smaller.

Therefore, by setting the condition that the difference change rate is equal to or greater than the threshold and identifying the cutoff energy when the condition is no longer satisfied, the identified cutoff energy becomes the cutoff energy suitable for the DFT calculation.

By determining the cutoff energy by the above-described method, the cutoff energy suitable for the DFT calculation may be determined by repetition of the SCF calculation performed in one DFT calculation, and the optimal cutoff energy may be calculated in a short time.

Note that the SCF calculation may be continuously performed even after the electron density obtained by the SCF calculation does no longer satisfy the predetermined condition. For example, in a case where the electron density obtained by the SCF calculation at the (N+1)-th time does not satisfy the predetermined condition, the processing unit 12 applies the second cutoff energy and executes the electron density calculations at the (N+2)-th time and thereafter. Thereby, calculation of the physical quantity (for example, the minimum value of the total energy) by the DFT calculation may be performed at the same time with the determination of the optimal cutoff energy. As a result, in a case where the DFT calculations under various initial conditions are performed using the determined cutoff energy, one DFT calculation may be performed at the same time with the determination of the cutoff energy, and the efficiency of the physical quantity calculation by the DFT may be achieved.

### Second Embodiment

A second embodiment shortens a calculation time of cutoff energy in a case of executing calculation of a molecular state of a substance by DFT with high-performance computing (HPC). Hereinafter, in the second embodiment, the cutoff energy is referred to as “Ecut”.

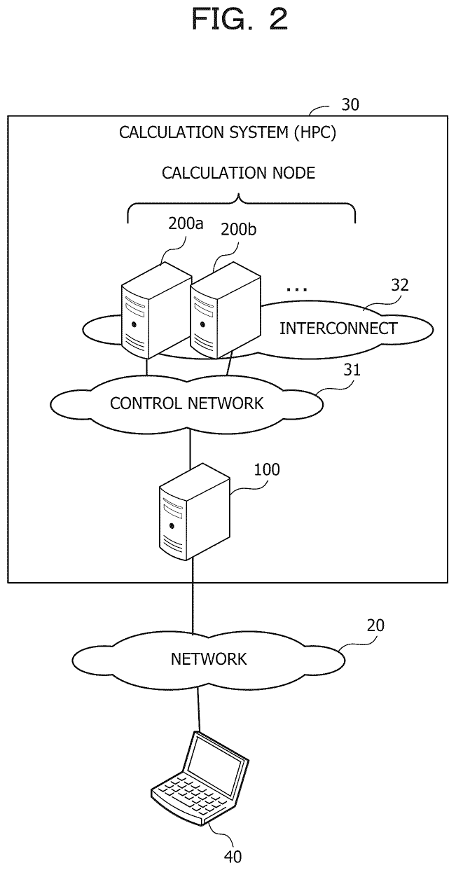

FIG. 2 is a diagram illustrating an example of a system configuration. A calculation system 30 that implements the HPC includes a control node 100 and a plurality of calculation nodes 200a, 200b, and the like. The plurality of calculation nodes 200a, 200b, and the like is coupled with each other by an interconnect 32 capable of performing high-speed communication. The interconnect 32 is, for example, a network in which processors are interconnected by a six-dimensional mesh/torus.

Each of the calculation nodes 200a, 200b, and the like is also coupled to a control network 31. The control node 100 is further coupled to the control network 31. The control node 100 is a computer that instructs the calculation nodes 200a, 200b, and the like to execute a job.

The control node 100 is coupled to a terminal 40 via a network 20. The terminal 40 is a computer that registers, in the control node 100, information regarding a job to be executed by the calculation nodes 200a, 200b, and the like based on a user operation. The control node 100 instructs the plurality of calculation nodes 200a, 200b, and the like to perform calculation by DFT according to the instruction from the terminal 40.

For example, a user may obtain a physical quantity such as total energy of an electronic system defined by the DFT by causing the calculation nodes 200a, 200b, and the like to execute the calculation of the state of the substance by the DFT.

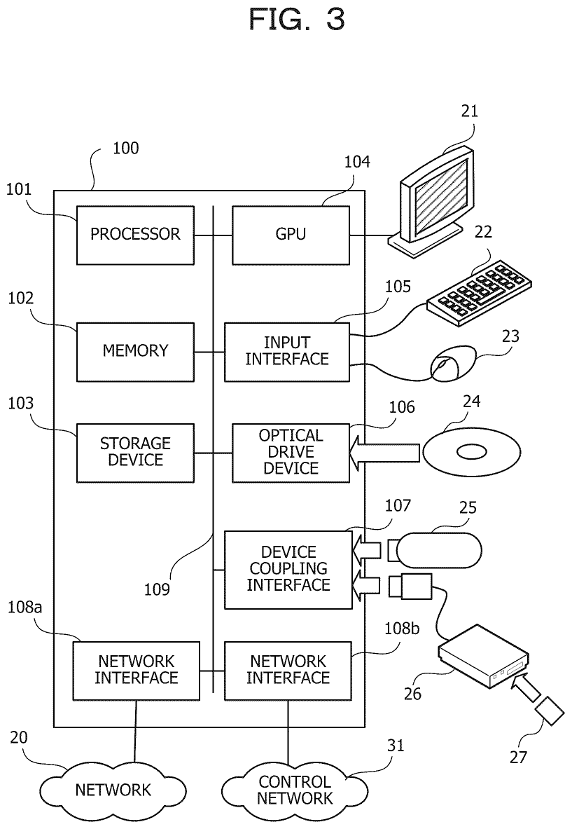

FIG. 3 is a diagram illustrating an example of a hardware configuration of the control node. The entire device of the control node 100 is controlled by a processor 101. A memory 102 and a plurality of peripheral devices are coupled to the processor 101 via a bus 109. The processor 101 may be a multiprocessor. The processor 101 is, for example, a central processing unit (CPU), a micro processing unit (MPU), or a digital signal processor (DSP). At least a part of functions implemented by execution of a program by the processor 101 may be implemented by an electronic circuit such as an application specific integrated circuit (ASIC) or a programmable logic device (PLD).

The memory 102 is used as a main storage device of the control node 100. The memory 102 temporarily stores at least a part of an operating system (OS) program and an application program to be executed by the processor 101. Furthermore, the memory 102 stores various types of data to be used in processing by the processor 101. As the memory 102, for example, a volatile semiconductor storage device such as a random access memory (RAM) is used.

Examples of the peripheral devices coupled to the bus 109 include a storage device 103, a graphics processing unit (GPU) 104, an input interface 105, an optical drive device 106, a device coupling interface 107, and network interfaces 108a and 108b.

The storage device 103 electrically or magnetically writes and reads data to and from a built-in recording medium. The storage device 103 is used as an auxiliary storage device of the control node 100. The storage device 103 stores an OS program, an application program, and various types of data. Note that, as the storage device 103, for example, a hard disk drive (HDD) or a solid state drive (SSD) may be used.

The GPU 104 is an arithmetic unit that performs image processing, and is also called a graphic controller. A monitor 21 is coupled to the GPU 104. The GPU 104 causes a screen of the monitor 21 to display an image in accordance with an instruction from the processor 101. Examples of the monitor 21 include a display device using organic electro luminescence (EL), a liquid crystal display device, and the like.

The input interface 105 is coupled with a keyboard 22 and a mouse 23. The input interface 105 transmits signals sent from the keyboard 22 and the mouse 23 to the processor 101. Note that the mouse 23 is an example of a pointing device, and another pointing device may also be used. Other pointing devices include a touch panel, a tablet, a touch pad, a track ball, and the like.

The optical drive device 106 reads data recorded on an optical disk 24 or writes data to the optical disk 24, using laser light or the like. The optical disk 24 is a portable recording medium in which data is recorded in a readable manner by reflection of light. Examples of the optical disk 24 include a digital versatile disk (DVD), a DVD-RAM, a compact disk read only memory (CD-ROM), a CD-recordable (R)/rewritable (RW), and the like.

The device coupling interface 107 is a communication interface for coupling peripheral devices to the control node 100. For example, a memory device 25 and a memory reader/writer 26 may be coupled to the device coupling interface 107. The memory device 25 is a recording medium equipped with a communication function with the device coupling interface 107. The memory reader/writer 26 is a device that writes data to a memory card 27 or reads data from the memory card 27. The memory card 27 is a card-type recording medium.

The network interface 108a is coupled to the network 20. The network interface 108a transmits and receives data to and from another computer such as the terminal 40 or a communication device via the network 20. The network interface 108b is coupled to the control network 31. The network interface 108b transmits and receives data to and from the calculation nodes 200a, 200b, and the like via the control network 31.

The network interfaces 108a and 108b are wired communication interfaces coupled to a wired communication device such as a switch or a router with a cable, for example. Furthermore, the network interfaces 108a and 108b may be wireless communication interfaces that are coupled to and communicate with a wireless communication device such as a base station or an access point with radio waves.

The control node 100 implements processing functions of the second embodiment by, for example, executing a program recorded in a computer-readable recording medium. The program in which processing content to be executed by the control node 100 is written may be recorded in various recording media. For example, the program to be executed by the control node 100 may be stored in the storage device 103. The processor 101 loads at least a part of the program in the storage device 103 into the memory 102, and executes the program. Furthermore, the program to be executed by the control node 100 may be recorded in a portable recording medium such as the optical disk 24, the memory device 25, or the memory card 27. The program stored in the portable recording medium becomes executable after being installed in the storage device 103 under the control of the processor 101, for example. Furthermore, the processor 101 may also read the program directly from the portable recording medium and execute the program.

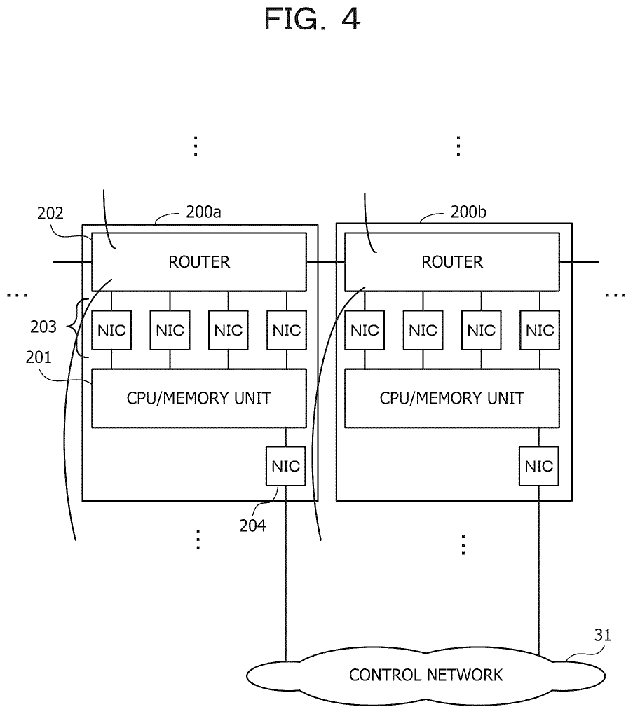

FIG. 4 is a diagram illustrating an example of a hardware configuration of the calculation node. The calculation node 200a includes a CPU/memory unit 201 and a router 202. The CPU/memory unit 201 and the router 202 are coupled by a plurality of communication interfaces (network interface cards (NICs)) 203. Furthermore, a NIC 204 for coupling to the control network 31 is also coupled to the CPU/memory unit 201.

The CPU/memory unit 201 includes a CPU having a plurality of cores and a memory. In the CPU/memory unit 201, a process (execution unit of processing) is generated for each core. In a case where the process for each core of the CPU/memory unit 201 performs synchronous processing with a process in another calculation node, the communication node communicates with the another calculation node 200b or the like via the router 202.

The router 202 communicates with, for example, a calculation node adjacent in each of three-dimensional directions. The router 202 transmits data to be transmitted from the CPU/memory unit 201 to another calculation node to a calculation node adjacent in a direction corresponding to the position in the interconnect 32 of the another calculation node. Furthermore, when receiving data from the adjacent calculation node to the process in the CPU/memory unit 201, the router 202 transmits the data to the CPU/memory unit 201. Moreover, in a case where the data received from the adjacent calculation node is data directed to another calculation node, the router 202 transmits the data to a calculation node adjacent in a direction corresponding to the position in the interconnect 32 of the another calculation node at transmission destination.

The other calculation nodes 200b and the like have a hardware configuration similar to the calculation node 200a. The calculation nodes 200a, 200b, and the like having the hardware configuration as illustrated in FIG. 4 are mesh/torus coupled in the three-dimensional directions, so that the three-dimensional mesh/torus interconnect 32 is achieved.

The control node 100 and the calculation nodes 200a, 200b, and the like having the above hardware configuration may implement the processing functions of the second embodiment. Note that the information processing device 10 described in the first embodiment may also be implemented by hardware configuration similar to the control node 100 or the calculation node 200a.

In the case of causing the calculation system 30 to execute quantum chemistry calculation by the DFT, the Ecut is determined in advance. There is a dependency relationship between the Ecut and total energy of a substance.

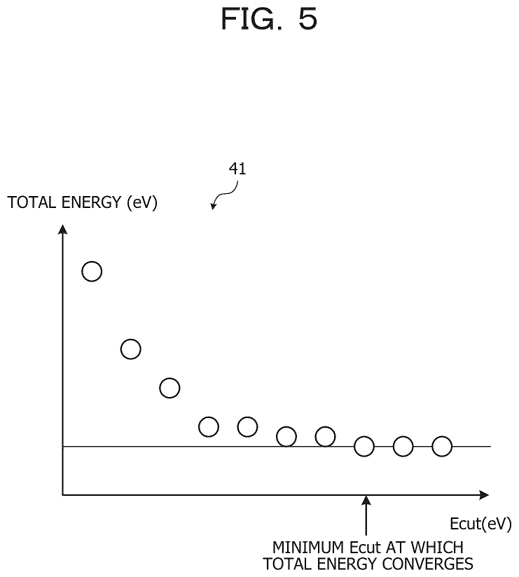

FIG. 5 is a diagram illustrating a relationship between the Ecut and the total energy. In a graph 41 illustrated in FIG. 5, the horizontal axis represents the Ecut, and the vertical axis represents the total energy of the DFT in the case of being calculated by the corresponding Ecut. The unit of the energy is eV.

As illustrated in the graph 41, the total energy decreases as the Ecut increases. That is, the total energy and the Ecut have dependency. However, when the Ecut increases to some extent or more, the total energy does not decrease (the total energy converges) even if the Ecut is increased. Furthermore, as the Ecut is increased, calculation is performed in consideration of a wave having a smaller wavelength in the DFT calculation, and a calculation amount increases. Therefore, the minimum Ecut at which the total energy converges is the optimal Ecut.

As described above, when the Ecut in the case of obtaining the state in which the total energy is minimized by the DFT calculation is excessively smaller than the optimal Ecut, sufficient calculation accuracy is not able to be obtained, and when the Ecut is excessively larger than the optimal Ecut, the calculation time is unnecessarily prolonged. Therefore, it is required to determine the Ecut to be applied to the DFT calculation as the Ecut that may cause the total energy to converge to the minimum value.

As a method for determining the optimal Ecut, for example, it is conceivable to execute the DFT calculation a plurality of times by changing (increasing or decreasing) the Ecut and extract the minimum Ecut in which the total energy value converges. However, in the total energy calculation by one DFT, the calculation is repeatedly performed at one or more points arranged in a space or a coordinate system until the electron density at all the points converges. For example, self-consistent SCF calculation is used to determine whether or not the electron density converges.

The point to be calculated for the electron density is, for example, a point indicating the position of each cell when the space in which the substance exists is divided into a large number of cells. As the number of divisions of the space increases, the number of points for calculating the electron density also increases accordingly. Therefore, in the case of accurate calculation, even one DFT calculation takes several hours.

The dependence relationship between the total energy and the Ecut may be understood from the relationship between the Ecut and a wave number. A wave function of the substance is expressed by the following formula.

$$\\ \begin{matrix}φ ( r ) \sim {\sum }_{k}^{\vert {k}_{max} \vert} {φ}_{k} \cdot exp [ ik \cdot r ] & ( 1 )\end{matrix}$$

r is a position vector indicating the position where the electron density is calculated. k is a wave number vector. Magnitude of the wave number vector indicates the wave number of the wave (how much vibration is completed per unit space). When the wavelength is λ, the wave number is 2π/λ. kₘₐₓ is the maximum value of the wave number. kₘₐₓ and Ecut has the following relationship.

|kₘₐₓ|²˜Ecut (2)

As illustrated in Formula (2), kₘₐₓ increases as Ecut increases. When kₘₐₓ increases, the number of wave numbers used in Formula (1) increases, and the calculation accuracy is improved. With the improvement of the calculation accuracy, the total energy may be brought close to the minimum value.

Here, in a case where the SCF calculation is repeated while the Ecut is fixed in one DFT calculation, only the total energy at the Ecut is obtained as a calculation result. Therefore, the calculation system 30 according to the second embodiment changes the Ecut every time one SCF calculation is performed in one DFT calculation, thereby enabling derivation of the optimal Ecut by one DFT calculation.

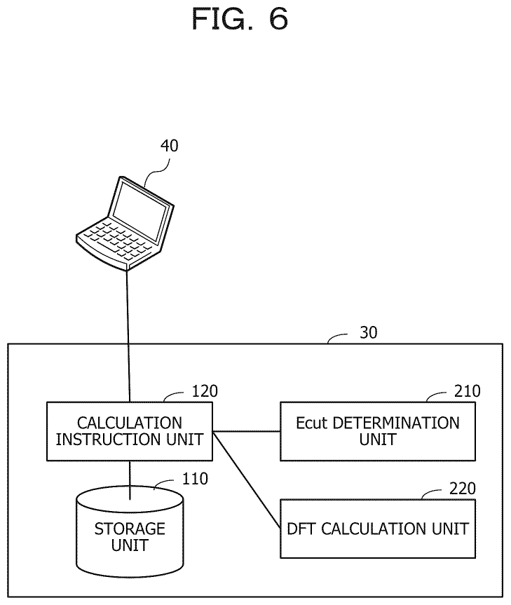

FIG. 6 is a block diagram illustrating an example of functions implemented by the calculation system. The calculation system 30 includes a storage unit 110, a calculation instruction unit 120, an Ecut determination unit 210, and a DFT calculation unit 220. The storage unit 110 and the calculation instruction unit 120 are functions of the control node 100, for example. The Ecut determination unit 210 and the DFT calculation unit 220 are functions that the calculation nodes 200a, 200b, and the like have, for example.

The storage unit 110 stores information regarding the substance to be calculated (atoms included in the substance, known atomic arrangements, and the like). When receiving an analysis instruction of physical properties of a predetermined substance or the like from the terminal 40, the calculation instruction unit 120 transmits an Ecut determination instruction for analyzing the substance to the Ecut determination unit 210.

The Ecut determination unit 210 determines the Ecut optimal for the DFT calculation. The DFT calculation unit 220 calculates the state of the substance by the DFT calculation to which the optimal Ecut is applied. For example, the DFT calculation unit 220 may perform first-principle calculation using the DFT and calculate a motion state of electrons such as the total energy. Furthermore, the DFT calculation unit 220 may also obtain a specific crystal structure (face-centered cubic structure, hexagonal close-packed structure, or the like) of the substance by the DFT calculation.

Note that the function of each element illustrated in FIG. 6 may be implemented by, for example, causing the computer to execute a program module corresponding to the element. For example, the control node 100 executes a program corresponding to the calculation instruction unit 120 to implement the calculation instruction unit 120. The calculation instruction unit 120 implemented by the control node 100 instructs one or more of the plurality of calculation nodes 200a, 200b, and the like to execute a job corresponding to the Ecut determination unit 210 or the DFT calculation unit 220. The instructed calculation node implements the Ecut determination unit 210 or the DFT calculation unit 220 by executing a program corresponding to the designated job.

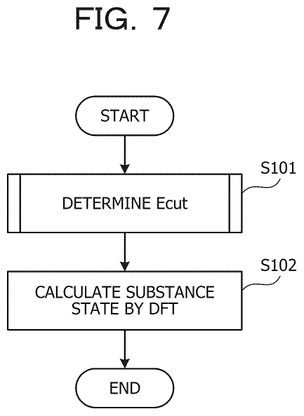

FIG. 7 is a flowchart illustrating an example of a substance state calculation procedure. Hereinafter, the processing illustrated in FIG. 7 will be described in line with step numbers.
[Step S101] The Ecut determination unit 210 performs Ecut determination processing. The Ecut determination unit 210 transmits the determined optimal Ecut value to the DFT calculation unit 220.
[Step S102] The DFT calculation unit 220 calculates the state of the substance by the DFT calculation to which the determined optimal Ecut is applied.

In the Ecut determination processing, the Ecut determination unit 210 executes the DFT calculation while varying the Ecut. For example, the Ecut determination unit 210 decreases the Ecut every time the SCF calculation is executed once until a predetermined Ecut change condition is satisfied. As the Ecut change condition, for example, a difference change rate of the electron density may be used.

The difference change rate of the electron density indicates a degree of change in a difference between the electron density estimated before the SCF calculation and the electron density calculated by the SCF calculation. For example, the difference in the electron density before and after the SCF calculation at the n-th time (n is a natural number) is defined as differences. The Ecut determination unit 210 compares differences with a representative value (mean, median, or the like) of differences difference) to differenceₙ₋₁.

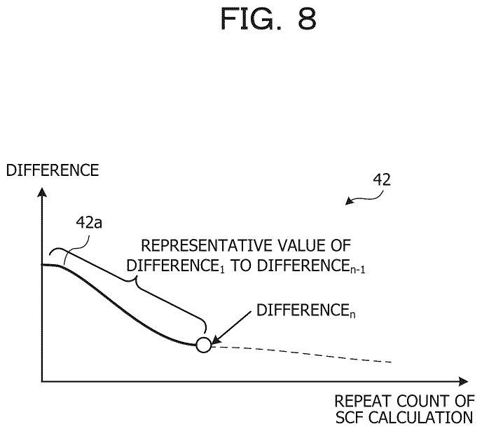

FIG. 8 is a diagram illustrating an example of differences and the representative value of difference₁ to differenceₙ₋₁. Graph 42 illustrates a change in the difference according to a repeat count of the SCF calculation. The horizontal axis of the graph 42 represents the repeat count of the SCF calculation, and the vertical axis represents the difference. As the repeat count of the SCF calculation increases, the difference decreases. The degree of decrease in the difference per SCF calculation decreases as the repeat count of the SCF calculation increases. For example, a curve 42a connecting points each indicating a value of the difference for each repeat count of the SCF calculation becomes close to horizontal when the repeat count of the SCF calculation increases to some extent or more.

In the case of obtaining the difference change rate of the SCF calculation at the n-th time, differenceₙ is compared with the representative value of difference₁ to differenceₙ₋₁. For example, the Ecut determination unit 210 sets the degree of change of differenceₙ from the representative value (mean, median, or the like) of difference₁ to differenceₙ₋₁ as the difference change rate in the SCF calculation at the n-th time. The difference change rate is expressed as follows.

difference change rateₙ=|((differenceₙ−the representative value of difference₁ to differenceₙ₋₁)/the representative value of difference₁ to differenceₙ₋₁| (3)

The Ecut determination unit 210 decreases the Ecut for each SCF calculation until such a difference change rate reaches a predetermined determination criterion (DTH). The determination criterion (DTH) of the difference change rate is set to, for example, “0.1” (10%). This determination criterion is an example of the threshold of the difference change rate described in the first embodiment.

A sufficiently large value capable of obtaining the minimum value of the total energy of the DFT is set as an initial value of the Ecut. While the Ecut is larger than the optimal value (the minimum Ecut at which the total energy converges), the electron density is calculated with high accuracy by the SCF, the difference in the electron density before and after the SCF calculation is large, and the difference change rate is a value larger than the determination criterion. When the Ecut becomes smaller than the optimal value, the difference change rate of the SCF becomes smaller than the determination criterion. Therefore, it is possible to obtain the optimal Ecut based on the difference change rate for each SCF calculation.

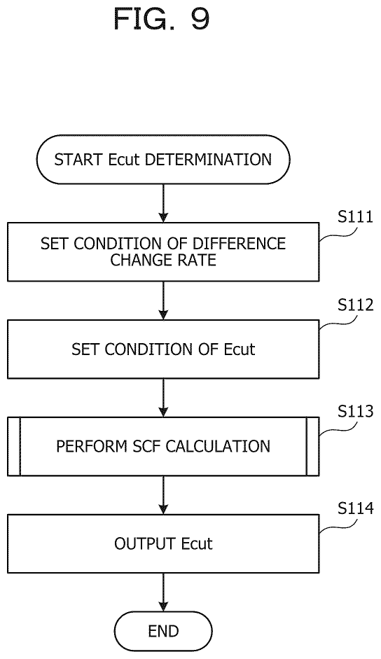

FIG. 9 is a flowchart illustrating an example of a procedure of the Ecut determination processing. Hereinafter, the processing illustrated in FIG. 9 will be described in line with step numbers.
[Step S111] The Ecut determination unit 210 sets the condition of the difference change rate. For example, the calculation instruction unit 120 transmits a job execution instruction including the condition of the difference change rate to the Ecut determination unit 210 based on an input from a user via the terminal 40. The Ecut determination unit 210 sets the instructed condition of the difference change rate as a job execution condition. For example, the Ecut determination unit 210 sets a type (which one of the mean, median, or the like is used) of the representative value of differenceₙ to differenceₙ₋₁ as the condition of the difference change rate. Furthermore, the Ecut determination unit 210 sets the determination criterion of the difference change rate.
[Step S112] The Ecut determination unit 210 sets the Ecut condition. For example, the calculation instruction unit 120 transmits the job execution instruction including the Ecut condition to the Ecut determination unit 210 based on the input from the user via the terminal 40. The Ecut determination unit 210 sets the instructed Ecut condition as the job execution condition. For example, the Ecut determination unit 210 sets the minimum value and the maximum value of a variation range of the Ecut as the Ecut condition. For example, the minimum value (Ecutₘᵢₙ) of the Ecut is 200 eV, and the maximum value (Ecutₘₐₓ) of the Ecut is 4000 eV.

Furthermore, the Ecut determination unit 210 sets a step size of the Ecut when reducing the Ecut. For example, the Ecut determination unit 210 sets the Ecut step size (Ecut_{delta}) to 50 eV.
[Step S113] The Ecut determination unit 210 executes the SCF calculation. As a result of the SCF calculation, the optimal Ecut is obtained.
[Step S114] The Ecut determination unit 210 outputs the optimal Ecut obtained by the SCF calculation.

In the SCF calculation in step S113, for example, the SCF calculation corresponding to one DFT calculation is repeatedly executed. Then, the Ecut to be applied to the next SCF calculation is determined every time the SCF calculation is executed once.

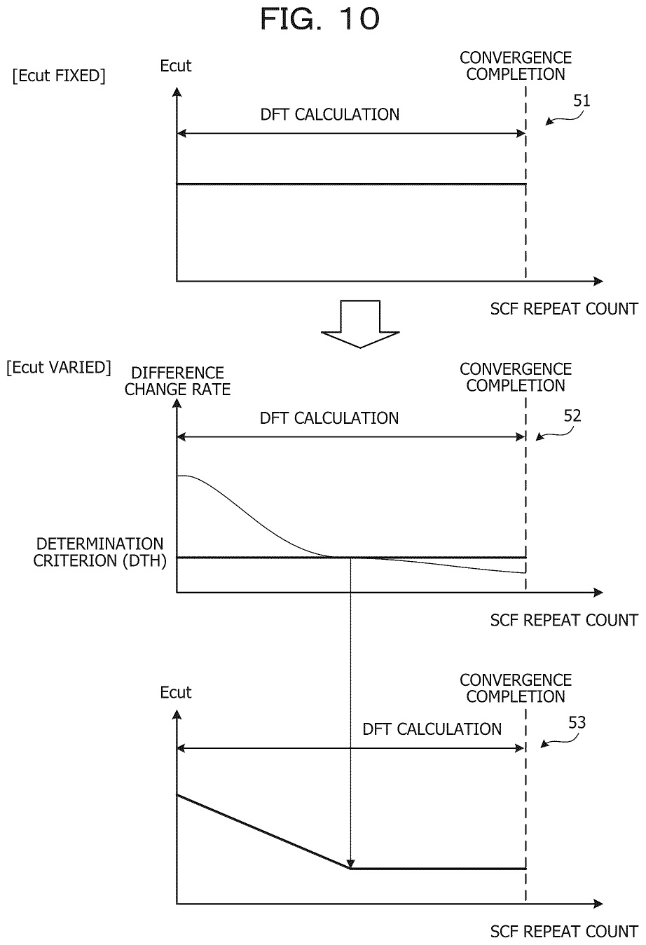

FIG. 10 is a diagram illustrating a comparative example of a case where Ecut is fixed and a case where Ecut is varied. A graph 51 illustrates the Ecut for each SCF calculation in the case where the Ecut is fixed in one DFT calculation. The horizontal axis of the graph 51 is the SCF repeat count, and the vertical axis represents the Ecut. In the case where Ecut is fixed, a common Ecut is used at each time of the SCF calculation.

A graph 52 illustrates the difference change rate for each SCF calculation in the case where the Ecut is varied in one DFT calculation. The horizontal axis of the graph 52 represents the SCF repeat count, and the vertical axis represents the difference change rate. The difference change rate decreases as the SCF repeat count increases. The Ecut determination unit 210 starts the SCF calculation with the Ecut with a value higher than the determination criterion (DTH). As a result, when the repeat count of the SCF calculation increases, the difference change rate eventually becomes equal to or less than the determination criterion (DHT).

A graph 53 illustrates the Ecut for each SCF calculation in the case where the Ecut is varied in one DFT calculation. The horizontal axis of the graph 53 represents the SCF repeat count, and the vertical axis represents the Ecut.

In the case of varying the Ecut, the initial value of the Ecut is the maximum value (Ecutₘₐₓ). By setting the maximum value of the Ecut to the value sufficiently larger than the expected optimal Ecut, the total energy in the current electronic state may be caused to converge to the minimum value in the state where the Ecut is the maximum value. While the difference change rate is above the determination criterion (DTH), the Ecut decreases by the Ecut step size (Ecut_{delta}) each time the SCF calculation is repeated.

The Ecut becomes a fixed value in the SCF calculation after the difference change rate becomes equal to or less than the determination criterion (DTH). The Ecut at the point of time of the end of one DFT calculation is the optimal value of the Ecut.

Next, an SCF calculation procedure for obtaining the Ecut optimal value will be described in detail.

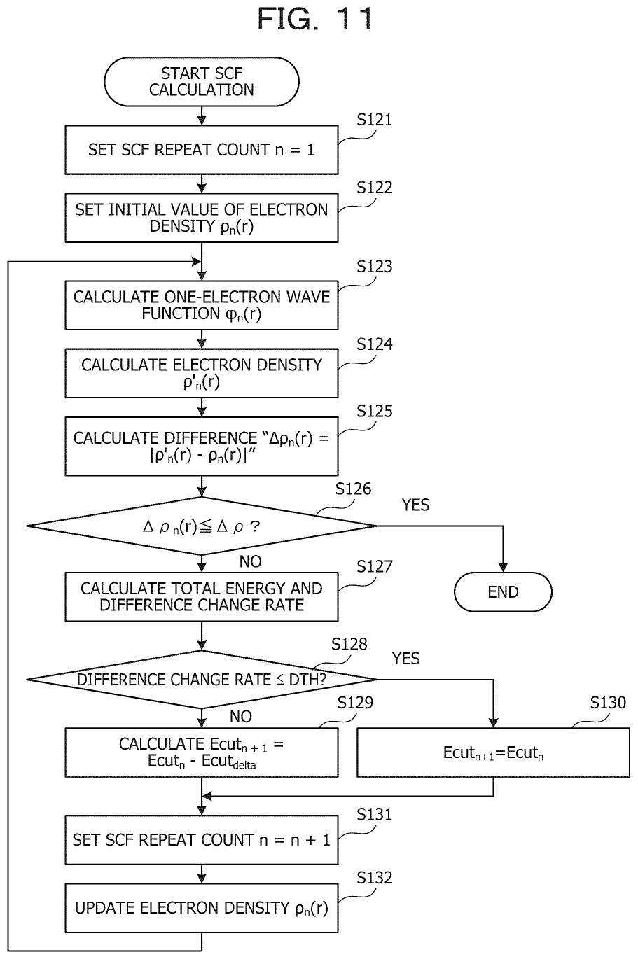

FIG. 11 is a flowchart illustrating an example of an SCF calculation procedure. Hereinafter, the processing illustrated in FIG. 11 will be described in line with step numbers.
[Step S121] The Ecut determination unit 210 sets the SCF repeat count n to 1 as the initial value.
[Step S122] The Ecut determination unit 210 sets the initial value of the electron density ρₙ(r) specified in advance for all the position vectors r.
[Step S123] The Ecut determination unit 210 calculates the one-electron wave function φₙ(r) (φ is one-stroke). For example, the Ecut determination unit 210 calculates the one-electron wave function φₙ(r) by solving a Kohn Sham formula. Ecutₙ is applied as the Ecut in this calculation. Ecut₁ in the SCF calculation at the first time is the Ecut maximum value (Ecutₘₐₓ).
[Step S124] The Ecut determination unit 210 calculates electron density ρ′ₙ(r) for each position vector based on the one-electron wave function φₙ(r).
[Step S125] The Ecut determination unit 210 calculates a difference Δρₙ(r) between the electron density ρₙ(r) and the electron density ρ′ₙ(r) for each position vector. The difference Δρₙ(r) may be calculated by, for example, “Δρₙ(r)=|ρ′ₙ(r)−ρₙ(r)|”.
[Step S126] The Ecut determination unit 210 determines whether or not the differences Δρₙ(r) for all the position vectors r are equal to or less than an allowable value Δρ. For example, the Ecut determination unit 210 extracts the maximum value of the differences Δρₙ(r) for the respective position vectors r. Then, when the extracted maximum value is equal to or less than the allowable value Δρ, the Ecut determination unit 210 determines that the differences Δρₙ(r) for all the position vectors r are equal to or less than the allowable value Δρ.

When the differences Δρₙ(r) for all the position vectors r are equal to or less than the allowable value Δρ, the Ecut determination unit 210 determines that the electron density has converged in the SCF calculation, and terminates the SCF calculation processing. Furthermore, when the difference Δρₙ(r) for at least one position vector r exceeds the allowable value Δρ, the Ecut determination unit 210 advances the processing to step S127.
[Step S127] The Ecut determination unit 210 calculates the total energy and the difference change rate for each position vector. For example, in the case of calculating the difference change rate, the Ecut determination unit 210 calculates the representative value of the difference Δρ₁(r) to the difference Δρₙ₋₁(r). Next, the Ecut determination unit 210 calculates an absolute value of the difference between the calculated representative value and the difference Δρₙ(r). Then, the Ecut determination unit 210 sets a value obtained by dividing the absolute value of the difference by the difference Δρ₁(r) as the difference change rate.
[Step S128] The Ecut determination unit 210 determines whether or not the difference change rates are equal to or less than the determination criterion (DTH) for all the position vectors r. When the difference change rates of all the position vectors r are equal to or less than the determination criterion (DTH), the Ecut determination unit 210 advances the processing to step S130. Furthermore, when the difference change rate of at least one position vector r is larger than the determination criterion (DTH), the Ecut determination unit 210 advances the processing to step S129.
[Step S129] The Ecut determination unit 210 calculates Ecutₙ₊₁ in the next SCF calculation. For example, the Ecut determination unit 210 sets a value obtained by subtracting the Ecut step size (Ecut_{delta}) from the current Ecutₙ as Ecutₙ₊₁ (Ecutₙ₊₁=Ecutₙ−Ecut_{delta}) Thereafter, the Ecut determination unit 210 advances the processing to step S131.
[Step S130] The Ecut determination unit 210 sets the current Ecutₙ as Ecutₙ₊₁ in the next SCF calculation (Ecutₙ₊₁=Ecutₙ).
[Step S131] The Ecut determination unit 210 adds 1 to the SCF repeat count n (n=n+1).
[Step S132] The Ecut determination unit 210 updates the electron density ρₙ(r). For example, the Ecut determination unit 210 sets the electron density ρ′ₙ₋₁(r) as the electron density ρₙ(r). Furthermore, the Ecut determination unit 210 may update the electron density ρₙ(r) to the electron density obtained by optimization calculation of the electron density based on the electron density ρₙ(r) or the electron density ρ′ₙ₋₁(r). After updating the electron density ρₙ(r), the Ecut determination unit 210 advances the processing to step S123.

In this way, in the case where the difference Δρₙ(r) becomes equal to or less than the allowable value Δρ at all the positions indicated by the position vectors, it is determined that the electron density has converged, and the Ecut at the point of time is determined to be the optimal Ecut. As a result, it is sufficient to perform the SCF calculation corresponding to one DFT calculation, and the calculation time for obtaining the optimal Ecut is greatly shortened.

The Ecut is gradually decreased in repetition of the SCF calculation. The calculation time of the DFT depends on the Ecut.

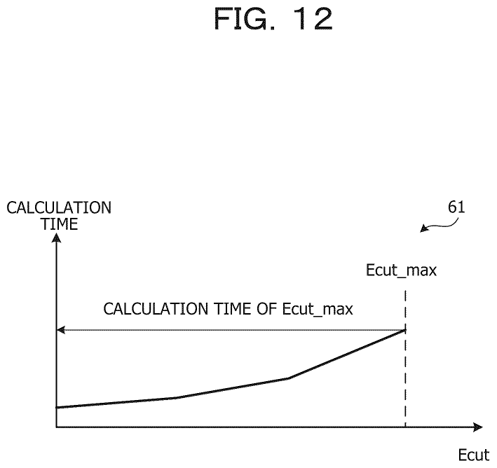

FIG. 12 is a diagram illustrating a relationship between the Ecut and the calculation time. A graph 61 in FIG. 12 illustrates the calculation time of the DFT according to the Ecut. The horizontal axis of the graph 61 represents the Ecut, and the vertical axis represents the calculation time. As illustrated in the graph 61, the calculation time increases as the Ecut increases. Here, the calculation time in the case of the maximum cutoff energy (Ecutₘₐₓ) is the maximum.

A speed-up rate of the optimal Ecut determination processing by varying the Ecut may be expressed by Formula (4) below with respect to the case where the DFT calculation is performed with the Ecut fixed during the DFT calculation.

The speed-up rate=the total calculation time (plurality of times) of the DFT calculation with the fixed Ecut/the calculation time of one DFT where the Ecut is varied (4)

The speed-up rate may be expressed by the following formula, assuming that the DFT calculation by the fixed Ecut is performed 10 times and an increase rate of the calculation time when the Ecut is increased by one stage is 1.2 times (linear).

$$\\ \begin{matrix}Speed - up rate = {\sum }_{i = 0}^{9} {( 1.2 )}^{- i} = 5. times & ( 5 )\end{matrix}$$

In this way, by determining the optimal Ecut by repetition of the SCF calculation with the variation of the Ecut, the optimal Ecut may be determined in a short time. For example, it is assumed that one DFT calculation takes 10 minutes in a case where DFT calculation of 100 atoms is performed. In this case, when attempting to obtain the optimal Ecut by ten DFT calculations with the fixed Ecut, the calculation takes 100 minutes. Meanwhile, by varying the Ecut and obtaining the optimal Ecut by one DFT calculation, the calculation may be performed in about ⅕ of the time (20 minutes). When the number of atoms further increases (for example, 400 or more), the time required for one DFT calculation further increases. Therefore, the effect of shortening the calculation time by varying the Ecut and obtaining the optimal Ecut by one DFT calculation is increased.

### Other Embodiments

In the second embodiment, the calculation is performed by the HPC, but when the number of atoms of the substance to be calculated is small, the calculation may be performed by a computer other than the HPC.

A formula other than Formula (3) may also be used to calculate the difference change rate. For example, the following Formula (6) may also be used as the difference change rate.

The difference change rate=(the current difference−the previous difference)/the previous difference (6)

By obtaining the change rate by Formula (6), the change rate may be obtained by the simpler formula.

Furthermore, the Ecut step size (Ecut_{delta}) indicating a decrease amount in decreasing the Ecut may be varied for each SCF calculation instead of the fixed value. For example, the Ecut step size (Ecut_{delta}) may be decreased as the difference change rate approaches the determination criterion (DTH).

While the embodiments have been exemplified thus far, the configuration of each unit illustrated in the embodiments may be replaced with another configuration having a similar function. Furthermore, any other components and steps may be added. Moreover, any two or more configurations (features) of the embodiments described above may be combined.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A non-transitory computer-readable recording medium storing a program for causing a computer to execute a process, the process comprising:
in repeat calculation of electron density of a substance by a self-consistent field method that uses a specific number of wave functions according to cutoff energy, executing a second electron density calculation at an (N+1)-th time (N is an integer greater than or equal to 1) by applying second cutoff energy of a value smaller than first cutoff energy applied to a first electron density calculation at an N-th time;
determining whether the electron density obtained by the second electron density calculation at the (N+1)-th time satisfies a predetermined condition; and
outputting the value of the second cutoff energy in a case where the condition is not satisfied.

2. The non-transitory computer-readable recording medium according to claim 1, the process further comprising:
executing electron density calculations at an (N+2)-th time and thereafter, by applying the second cutoff energy in the case where the condition is not satisfied.

3. The non-transitory computer-readable recording medium according to claim 2, the process further comprising:
executing the electron density calculation at the (N+2)-th time, by applying third cutoff energy obtained by reducing a predetermined value from the second cutoff energy in a case where the condition is satisfied.

4. The non-transitory computer-readable recording medium according to claim 1, the process further comprising:
calculating a difference change rate of the electron density based on first differenceₙ and a second difference, each of the first differenceₙ indicating a difference between the electron density set at start of calculation and the electron density obtained as a calculation result in each electron density calculation up to the N-th time, the second difference indicating a difference between the electron density set at start of calculation in the second electron density calculation at the (N+1)-th time and the electron density obtained as a calculation result thereof; and
determining whether the condition is satisfied based on the difference change rate.

5. The non-transitory computer-readable recording medium according to claim 4, the process further comprising:
calculating a difference between a representative value of the first differenceₙ for respective electron density calculations up to the N-th time and the second difference as the difference change rate; and
determining that the condition is not satisfied in a case where the difference change rate is equal to or less than a predetermined threshold.

6. The non-transitory computer-readable recording medium according to claim 1, the process further comprising:
calculating a predetermined physical quantity of the substance based on a density functional theory, by applying the output second cutoff energy.

7. A cutoff energy determination method, comprising:
in repeat calculation of electron density of a substance by a self-consistent field method that uses a specific number of wave functions according to cutoff energy, executing by a computer a second electron density calculation at an (N+1)-th time (N is an integer greater than or equal to 1) by applying second cutoff energy of a value smaller than first cutoff energy applied to a first electron density calculation at an N-th time;
determining whether the electron density obtained by the second electron density calculation at the (N+1)-th time satisfies a predetermined condition; and
outputting the value of the second cutoff energy in a case where the condition is not satisfied.

8. An information processing device, comprising:
a memory; and
a processor coupled to the memory and the processor configured to:
in repeat calculation of electron density of a substance by a self-consistent field method that uses a specific number of wave functions according to cutoff energy, execute a second electron density calculation at an (N+1)-th time (N is an integer greater than or equal to 1) by applying second cutoff energy of a value smaller than first cutoff energy applied to a first electron density calculation at an N-th time;
determine whether the electron density obtained by the second electron density calculation at the (N+1)-th time satisfies a predetermined condition; and
output the value of the second cutoff energy in a case where the condition is not satisfied.
